(19) United States
(12) Patent Application Publication
(10) Pub. No.: US 2024/0049959 A1
(43) Pub. Date: Feb. 15, 2024

(54) METHODS AND SYSTEMS FOR SIGNAL FEATURE ANALYSIS

(71) Applicants: UNITED STATES GOVERNMENT AS REPRESENTED BY THE DEPARTMENT OF VETERANS AFFAIRS, Washington, DC (US); GEORGIA TECH RESEARCH CORPORATION, Atlanta, GA (US)

(72) Inventor: Andrew Feola, Washington, DC (US)

(21) Appl. No.: 18/269,473

(22) PCT Filed: Dec. 23, 2021
(86) PCT No.: PCT/US2021/065076

§ 371 Date: Jun. 23, 2023

Related U.S. Application Data
(60) Provisional application No. 63/130,156, filed on Dec. 23, 2020.

(51) Int. Cl. A61B 3/00; A61B 5/398

(52) U.S. Cl. CPC A61B 3/0008; A61B 5/398

(57) ABSTRACT

Methods, systems, and apparatuses are described for causing light to be emitted, determining a physiological response, receiving, based on the physiological response, a signal, determining one or more signal features, and determining, based on the one or more signal features, a physiological condition.

### CROSS-REFERENCE TO RELATED APPLICATION

This application claims priority to, and the benefit of, U.S. Provisional Application No.: 63/130,156, filed Dec. 23, 2020, the entirety of which is hereby incorporated by reference.

### BACKGROUND

Eye diseases often result in localized dysfunction of the retina. In a clinical setting, electroretinography is a useful, non-invasive procedure for determining spatial differences in retinal activity in which electrical potentials generated by the retina of the eye are measured upon exposing the retina to a light stimulus. In conducting an electroretinography and generating an electroretinogram (ERG), an electrode is positioned on the cornea of a patient's eye and a second electrode, usually referred to as an “indifferent” electrode is positioned to complete an electrical connection with the patient's upper anatomy. The patient's eye is exposed to a light stimulus that causes a physiological response (an electrochemical signal). However, there can be inter-observer variability in the data analysis. Further, ERG analysis requires expert understanding of the ERG response to provide accurate analysis and interpretation. This often becomes time-intensive due to the number of flash intensities, subjects, and time points. Thus, a new method and system are required.

### SUMMARY

Methods, systems, and apparatuses are described for determining features associated with a waveform caused by analyzing the bioelectric activity of the retina from potentials recorded at the cornea. This method provides an array of electrodes, and places the electrodes in electrical contact with the cornea. While illuminating the eye so as to cause retinal activity, measurements are made, via the array of electrodes, of the electrophysiological potentials at the cornea in response to the illumination. The method includes solving for retinal information based on the electrophysiological potentials made at the cornea based on a raw waveform. In one example, the method uses standard full-field stimuli in conjunction with a corneal multi-electrode array. The subject is exposed to a light stimulus, causing an electrophysiological response which generates a raw waveform to be analyzed. In addition to the corneal electrode array, an appropriate analysis or source modeling of the collected data provides information regarding the location and extent of retinal dysfunction. That is to say, certain features of the signal are associated with particular types of cells (usually found in localized regions) within the eye. Results are achieved using standard electrophysiology amplifiers and digital data acquisition systems. The present methods, systems, and apparatuses provide a semi-automated analysis program to perform non-subjective and repeatable feature identification (“marking”) of the ERG waveform. This program is capable of marking the standard a-wave (photoreceptor layers), b-wave (inner retina), and oscillatory potentials or “OPs” (amacrine cells/inner retina) response. Further, the present methods, systems, and apparatuses provide for advanced ERG analysis (e.g. waveform modeling and power analysis).

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

To easily identify the discussion of any particular element or act, the most significant digit or digits in a reference number refer to the figure number in which that element is first introduced.

FIG. 1 show an example system;

FIGS. 2A-2B show example diagrams of an ERG device and an eye;

FIGS. 3A-3C show example diagrams of an eye and waveforms;

FIG. 4 shows an example diagram of an ERG device and an eye;

FIGS. 5A-5B show an example lighting device;

FIG. 6 shows an example ERG device;

FIG. 7 shows an example method;

FIG. 8 shows an example method;

FIG. 9 shows an example method;

FIG. 10 shows an example method;

FIG. 11 shows an example method;

FIG. 12 shows an example table of physiological conditions; and

FIG. 13 shows an example system.

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application, including, but not limited to, steps in disclosed methods. Thus, if there are a variety of additional steps that can be performed, it is understood that each of these additional steps can be performed with any specific embodiment or combination of embodiments of the disclosed methods.

The present methods and systems may be understood more readily by reference to the following detailed description of preferred embodiments and the examples included therein and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized, including hard disks, CD-ROMs, optical storage devices, or magnetic storage devices.

Embodiments of the methods and systems are described below with reference to block diagrams and flowchart illustrations of methods, systems, apparatuses, and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Hereinafter, various embodiments of the present disclosure will be described with reference to the accompanying drawings. As used herein, the term “user” may indicate a person who uses an electronic device or a device (e.g., an artificial intelligence electronic device) that uses an electronic device.

The present disclosure provides a method used to calculate, approximate, or infer information about electrophysiological activity in the retina, based on measurements of electrical potentials made at the anterior surface of the eye. This method may include appropriate adaptations of any of the varied techniques developed for functional brain mapping based on electroencephalographic recordings, or those developed for mapping of cardiac activity based on measurements of cardiac potentials made at the surface of the heart or the torso, or any combination of elements of these techniques applied to solving for retinal potentials or currents based on knowledge of eye surface potentials. With this computational method, retinal activity is determined from measurements of eye surface potentials via an electrode array, as set out herein.

The present disclosure is also directed to the use of known photic stimuli, which are designed to selectively elicit responses from specific cell types or functional pathways in the retina. These stimuli are used in conjunction with an array of eye surface measurement electrodes as described above, such that differences in function of these cell types or functional pathways can be obtained.

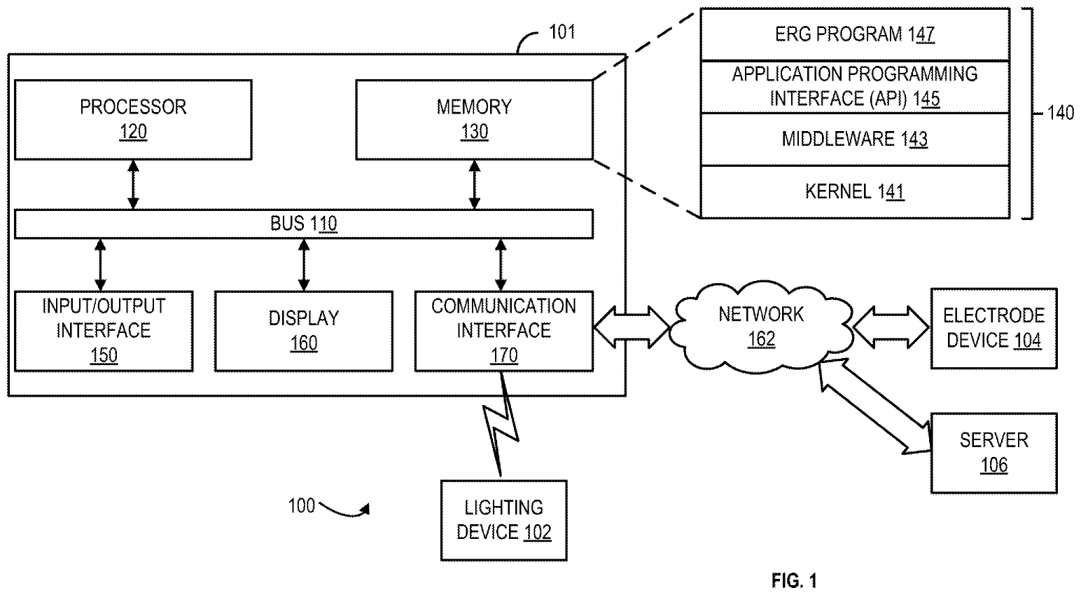

FIG. 1 illustrates a network environment including an electronic device configured for ERG signal analysis according to various embodiments. Referring to FIG. 1, an ERG device 101 in a network environment 100 is disclosed according to various exemplary embodiments. The ERG device 101 may include a bus 110, a processor 120, a memory 130, an input/output interface 150, a display 160, and a communication interface 170. In a certain exemplary embodiment, the ERG device 101 may omit at least one of the aforementioned constitutional elements or may additionally include other constitutional elements. The ERG device 101 may be, for example, a computer, a mobile phone, a tablet, a laptop, a desktop computer, a smartwatch, or the like. The ERG device 101 may comprise or otherwise be connected to a signal processor (e.g., a signal sensing and recording device) capable of detecting and amplifying signals from an electrode device 104 comprising one or more electrodes that preferably includes an amplifier and is capable of detecting and amplifying an electrical potential signal from each electrode of the one or more electrodes. The signal processor preferably is capable of processing the electric potential signals obtained from ERG measurements in a form suitable for data analysis. The signal processor may include or can be interfaced with a data storage device (e.g., random access memory, hard drive storage, and the like) and optionally includes or can be interfaced with a display device (e.g., user interface) for displaying some or all of the recorded electrical potentials, e.g., in the form of numerical tables, individual electroretinographs, or as a map of retinal activity, as desired. The electrical potential data recorded from each electrode is stored in a manner such that the data can be individually accessed and/or analyzed, and which can be combined with electric potential data from one or more other electrodes, as desired, e.g., for noise reduction purposes. In some embodiments, a computer is programmed to generate a map of retinal activity from the electric potential data.

The bus 110 may include a circuit for connecting the aforementioned constitutional elements 110 to 170 to each other and for delivering communication (e.g., a control message and/or data) between the aforementioned constitutional elements.

The processor 120 may include one or more of a Central Processing Unit (CPU), an Application Processor (AP), and a Communication Processor (CP). The processor 120 may control, for example, at least one of other constitutional elements of the ERG device 101 and/or may execute an arithmetic operation or data processing for communication. The processing (or controlling) operation of the processor 120 according to various embodiments is described in detail with reference to the following drawings.

The memory 130 may include a volatile and/or non-volatile memory. The memory 130 may store, for example, a command or data related to at least one different constitutional element of the ERG device 101. According to various exemplary embodiments, the memory 130 may store a software and/or a program 140. The program 140 may include, for example, a kernel 141, a middleware 143, an Application Programming Interface (API) 145, and/or an ERG application program (e.g., “application” or “mobile app”) 147, or the like. The ERG program 147 may be configured for controlling one or more functions of the ERG device 101 and/or an external device (e.g., an electrode device and/or a lighting device). At least one part of the kernel 141, middleware 143, or API 145 may be referred to as an Operating System (OS). The memory 130 may include a computer-readable recording medium having a program recorded therein to perform the method according to various embodiment by the processor 120.

The ERG program 147 may be configured to generate an ERG. An electroretinogram (ERG) is a recording of one or more bioelectric signals arising in the retina, and is recorded in response to a light stimulus. In a clinical setting, an ERG is recorded using non-invasive means, where the active electrode may be integral to, for example, a contact lens that allows an unobstructed view of the stimulus source. While a contact lens is referenced herein, it is to be understood that the electrode(s) (e.g., electrode array) may not be integrated in a contact lens but rather may contact the cornea through any appropriate means. A corneal ERG is a potential reflecting the summed contribution of all retinal cells responsive to the stimulus. The most typical type of stimulus is a brief (<1 ms) full-field flash, wherein the stimulus has constant luminance across the entire visual field. The ERG program 147 may be configured as a semi-automated analysis program to perform non-subjective and repeatable feature identification (“marking”) of the ERG waveform. This program is capable of marking the standard a-wave (photoreceptor layers), b-wave (inner retina), and oscillatory potentials (amacrine cells/inner retina) response. Further, the systems and methods described herein include advanced ERG analysis (e.g. waveform modeling and power analysis).

The ERG program 147 may be in communication with (e.g., via the communication interface 170) one or more of a lighting device 102, an electrode device 104, and/or a server 106. The lighting device 102 may be configured for retinal illumination. Retinal illumination during an ERG may be conducted in a number of ways. For example, a first set of electroretinographic readings may be taken in normal room light. In a second step, the lights may be dimmed for a significantly long period of time (e.g., on the order of 20 minutes), and readings are taken while the subject's retina is exposed to a light source. The ERG can also be performed under light (photopic) conditions to get a different response that nonetheless generates a waveform to be processed as described further herein. That is, after prolonged period in a dark environment, electrophysiological readings are taken at the onset of retinal exposure to light, and for a time period shortly thereafter. For example, after a sufficient time for adaptation of the retina to the dark environment has passed, a bright flash may be directed to the subject's retina with electroretinogram readings being taken. Each electroretinogram reading will differ depending upon the light conditions to which the patient's retina is subjected. However, standard responses have been established for each type of test and various useful conclusions can be drawn from excursions from such standardized data. In each test, the retinal response to each illumination is typically in the form of a voltage versus time waveform. Different types of waveforms have been defined for normal retinal responses. It is expected in a healthy subject, for example, that an electroretinogram shows a-wave (initial negative deflection associated with photoreceptors), b-wave (positive deflection associated with photoreceptors, bipolar, amacrine, and Muller cells such as Muller glia), and Oscillatory Potentials (OPs) patterns normal in shape and duration, with appropriate increases in electrical activity as the stimulus intensity is increased.

The electrode device 104 may be configured to determine (e.g., measure, detect) one or more corneal potentials. The electrode device 104 may be positioned so as to contact, respectively, the cornea and the upper anatomy of a patient. The term “patient” may refer to either or both of an animal subject or a human subject. The one or more electrodes may, for example, be mounted on a contact lens for convenient application in an outpatient setting. The one or more electrodes may comprise Burian-Allen electrodes, Dawson-Trick-Litzkow electrodes, Jet electrodes, skin electrodes, mylar electrodes, Cotton-Wick electrodes, Hawlina-Konec Electrodes, combinations thereof, and the like. Similarly, the one or more electrodes may be positioned such that one electrode of the one or more electrodes contacts the cornea and another electrode of the one or more electrodes contacts, for example, the forehead, earlobe, or another part of anatomy. Such an electrode typically measures summed activity from the entire retina. In general, the electrical changes caused by the different major cell types of the retina (e.g., rod and cone photoreceptors, bipolar cells, horizontal cells, amacrine cells, ganglion cells, and Muller cells) tend to overlap in time, thus complex and varying waveforms are observed (e.g., a raw waveform comprising a plurality of waves). The most prominent wave is the b-wave and the height of this wave can provide an indication of the subject's sensitivity to the illumination source. Tests can be conducted with illumination sources of different spectral content, intensity, kinetics, spatial patterns and spatial contrast, etc., and the results can be studied to determine the state of the subject's ocular health.

The kernel 141 may control or manage, for example, system resources (e.g., the bus 110, the processor 120, the memory 130, etc.) used to execute an operation or function implemented in other programs (e.g., the middleware 143, the API 145, or the application program 147). Further, the kernel 141 may provide an interface capable of controlling or managing the system resources by accessing individual constitutional elements of the ERG device 101 in the middleware 143, the API 145, or the application program 147.

The middleware 143 may perform, for example, a mediation role so that the API 145 or the application program 147 can communicate with the kernel 141 to exchange data.

Further, the middleware 143 may handle one or more task requests received from the application program 147 according to a priority. For example, the middleware 143 may assign a priority of using the system resources (e.g., the bus 110, the processor 120, or the memory 130) of the ERG device 101 to at least one of the application programs 147. For instance, the middleware 143 may process the one or more task requests according to the priority assigned to the at least one of the application programs, and thus may perform scheduling or load balancing on the one or more task requests.

The API 145 may include at least one interface or function (e.g., instruction), for example, for file control, window control, video processing, or character control, as an interface capable of controlling a function provided by the application 147 in the kernel 141 or the middleware 143.

For example, the input/output interface 150 may play a role of an interface for delivering an instruction or data input from a user or a different external device(s) to the different constitutional elements of the ERG device 101. Further, the input/output interface 150 may output an instruction or data received from the different constitutional element(s) of the ERG device 101 to the different external device(s).

The display 160 may include various types of displays, for example, a Liquid Crystal Display (LCD) display, a Light Emitting Diode (LED) display, an Organic Light-Emitting Diode (OLED) display, a MicroElectroMechanical Systems (MEMS) display, or an electronic paper display. The display 160 may display, for example, a variety of contents (e.g., text, image, video, icon, symbol, etc.) to the user. The display 160 may include a touch screen. For example, the display 160 may receive a touch, gesture, proximity, or hovering input by using a stylus pen or a part of a user's body.

In an embodiment, the display 160 may be configured for displaying a user interface. The user interface may be configured to receive inputs. For example, the display 160 may comprise a touchscreen. Via the user interface, a user may execute an ERG program. The emitted light may comprise one or more lighting parameters. The one or more lighting parameters may comprise one or more flicker frequencies, intensities (e.g., luminance), colors (e.g., chroma), patterns, location within a field of view (e.g., central or peripheral) combinations thereof, and the like.

The communication interface 170 may establish, for example, communication between the ERG device 101 and the external device (e.g., a lighting device 102, electrode device 104, or a server 106). For example, the communication interface 170 may communicate with the external device (e.g., the electrode device 104 or the server 106) via a network 162. The network 162 may make use of both wireless and wired communication protocols.

For example, as a wireless communication protocol, the wireless communication may use at least one of Long-Term Evolution (LTE), LTE Advance (LTE-A), Code Division Multiple Access (CDMA), Wideband CDMA (WCDMA), Universal Mobile Telecommunications System (UMTS), Wireless Broadband (WiBro), Global System for Mobile Communications (GSM), other cellular technologies, combinations thereof, and the like. Further, the wireless communication may include, for example, a near-distance communication protocol 164. The near-distance communication protocol 164 may include, for example, at least one of Wireless Fidelity (WiFi), Bluetooth, Near Field Communication (NFC), Global Navigation Satellite System (GNSS), and the like. According to a usage region or a bandwidth or the like, the GNSS may include, for example, at least one of Global Positioning System (GPS), Global Navigation Satellite System (Glonass), Beidou Navigation Satellite System (hereinafter, “Beidou”), Galileo, the European global satellite-based navigation system, and the like. Hereinafter, the “GPS” and the “GNSS” may be used interchangeably in the present document. The wired communication may include, for example, at least one of Universal Serial Bus (USB), High Definition Multimedia Interface (HDMI), Recommended Standard-232 (RS-232), power-line communication, Plain Old Telephone Service (POTS), and the like. The network 162 may include, for example, at least one of a telecommunications network, a computer network (e.g., LAN or WAN), the internet, and a telephone network.

For example, the ERG program may cause a light to be emitted via the lighting device 102. The lighting device 102 may be configured for emitting light at a frequency ranging from about 10 Hz to about 60 Hz. The display 160 may be configured for adjusting any of the lighting parameters. For example, via the display 160, a user may cause the ERG program to increase or decrease the frequency, increase or decrease the intensity, change a color, change a pattern, combinations thereof, and the like. In an embodiment, the lighting device 102 may comprise one or more light emitting diodes (LED), one or more liquid crystal displays (LCD), one or more Cold Cathode Fluorescent Lamps (CCFL), combinations thereof, and the like. The application program 147 may be configured to communicate with the lighting device 102 via the network 164 to control the one or more lighting parameters.

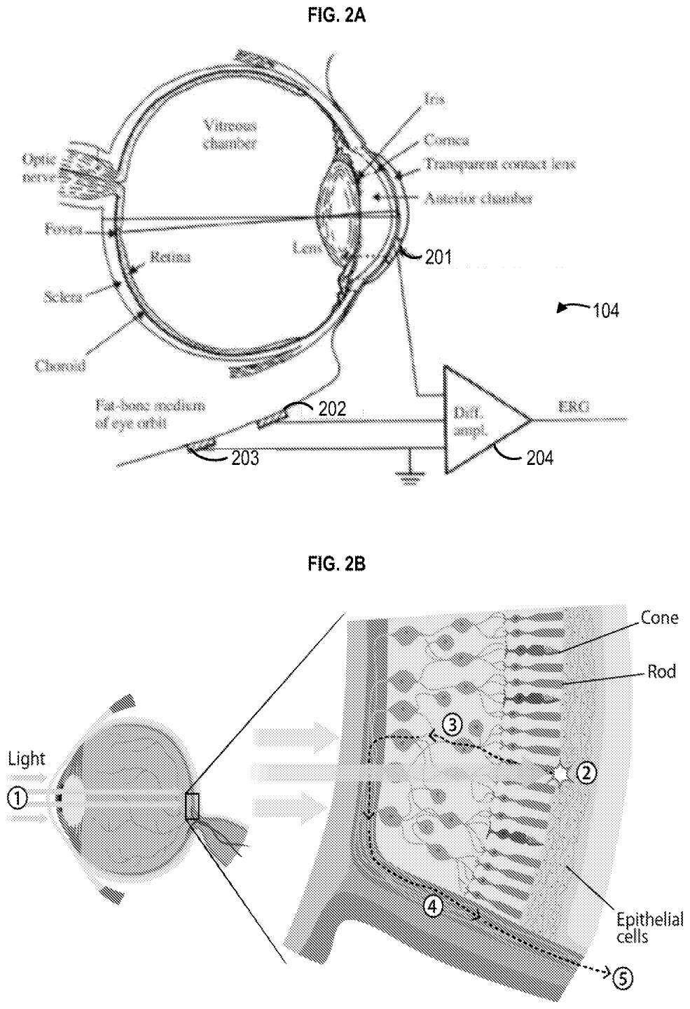

In an embodiment, the electrode device 104 may comprise a corneal contact module comprising an array of electrodes as described further herein. It should be appreciated that the array of electrodes may be at least translucent, so that it can transmit at least some light from an external illumination source to the retina, but does not necessarily need to be transparent. A translucent array may preclude formation of a visual image on the retina, but still allows for sufficient light from the stimulus source to reach the retina and elicit a bioelectric response. Light scattering by a partially opaque or translucent corneal contact module electrode array could be advantageous in some instances in the multi-electrode electroretinography (meERG) techniques of the invention by providing a uniform illumination of the retina, thereby simplifying the design of the stimulating light source. For example, the electrode array can be formed from a translucent, cloudy material, or alternatively, the array can comprise very narrow (fine) or thin conductive elements that transmit a sufficient amount of light, while not necessarily being optically clear and transparent. Likewise, the electrode array may simply contact the cornea and not be disposed on a film or substrate. The array of electrodes is positioned about the subject's eye in a manner conducive to contacting the subject's cornea. If desired, the subject's sclera can also be contacted. In an embodiment, the electrode device 104 may be a handheld device (e.g., the ERG device of FIG. 2.)

According to one exemplary embodiment, the server 106 may include a group of one or more servers. According to various exemplary embodiments, all or some of the operations executed by the ERG device 101 may be executed in a different one or a plurality of electronic devices (e.g., the lighting device 102, the electrode device 104, or the server 106). The electrode device 104 may be the corneal contact module. According to one exemplary embodiment, if the ERG device 101 needs to perform a certain function or service either automatically or at a request, the ERG device 101 may request at least some parts of functions related thereto alternatively or additionally to a different electronic device (e.g., the lighting device 102, the electrode device 104, or the server 106) instead of executing the function or the service autonomously. The different electronic device (e.g., the lighting device 102, the electrode device 104, or the server 106) may execute the requested function or additional function and may deliver a result thereof to the ERG device 101. The ERG device 101 may provide the requested function or service either directly or by additionally processing the received result. For this, for example, a cloud computing, distributed computing, or client-server computing technique may be used.

FIG. 2A is a diagram illustrating placement of one embodiment of the electrode device 104 on the eye of a patient. As seen in FIG. 2A, the eye comprises an iris, a cornea, a lens, an anterior chamber in front of the lens, a vitreous chamber behind the lens, a retina at the back of the vitreous chamber, a fovea, a sclera, a choroid, and an optic nerve leading to the brain. The embodiment of the electrode device 104 may comprise one or more contact lens electrodes 201, one or more reference electrodes 202, one or more ground electrodes 203, one or more amplifiers 204, and may be in communication with the ERG device 101. During an ERG session, the one or more contract lens electrode 201 may be disposed in or on a contact lens (e.g., a transparent contact lens). Although FIG. 2A shows one or more contact lens electrodes 201, it is to be understood that the systems and methods described herein may incorporate one or more ERG electrodes disposed directly on the eye (e.g., not on a contact lens), or any other suitable configuration. The one or more electrodes may comprise Burian-Allen electrodes (consists of an annular ring of stainless steel surrounding a polymethylmethacrylate (PMMA) contact-lens core. BA electrodes incorporate a lid speculum, which helps to minimize eye blinks/closure. BA lenses are reusable and are available in sizes ranging from pediatric to adult), Dawson-Trick-Litzkow electrodes (low-mass conductive silver/nylon thread, DTL electrodes are disposable and are typically more comfortable for the patients, as compared to other corneal electrodes), Jet electrodes (disposable plastic lens with a gold-plated peripheral circumference), skin electrodes (may be used as a replacement for corneal electrodes by placing an electrode on the skin over the infraorbital ridge near lower eyelid. ERG amplitudes tend to be small and noisy, but skin electrodes are better-tolerated in pediatric populations), mylar electrodes (aluminized or gold-coated Mylar), Cotton-Wick electrodes (Burian-Allen electrode shell fitted with a cotton wick, which is useful for minimizing light-induced artifacts), Hawlina-Konec Electrodes (Teflon-insulated thin metal wire (silver, gold, platinum) with three central windows, 3 mm in length, molded to fit into the lower conjunctival sac), combinations thereof, and the like.

The one or more contact lens electrodes 201 may be configured to determine (e.g, detect, measure, receive) one or more corneal potentials. The one or more corneal potentials may be associated with, an ERG signal. The one or more corneal potentials, and the ERG signals (and/or components such as “waves” thereof) may be associated with one or more parts of the eye. For example, as seen in FIG. 2B, the retina may comprise one or more rods, one or more cones, and one or more epithelial cells. FIG. 2B shows an example of how a patient's eye may react to light. For example, at step 1, light enters the eye through the lens and passes through the vitreous chamber until it strikes the cells at the back of the eye (e.g., the retina). In particular, at step 2, the light may strike one or more epithelial cells and stimulate one or more rods or one or more cones. Rods are responsible for vision at low light levels (e.g., scotopic vision). They do not mediate color vision, and have a low spatial acuity. Cones are active at higher light levels (e.g., photopic vision), are capable of color vision and are responsible for high spatial acuity. The central fovea is populated exclusively by cones. There are 3 types of cones: the short-wavelength sensitive cones, the middle-wavelength sensitive cones and the long-wavelength sensitive cones or S-cone, M-cones, and L-cones for short. Both rods and cones are operational at mid-level lighting (e.g, mesopic vision). At step 3, the one or more rods and/or the one or more cones, in response to the light stimulus, may send one or more electrochemical signals to the optic nerve for transport out of the eye to the brain (e.g., at steps 4 and 5).

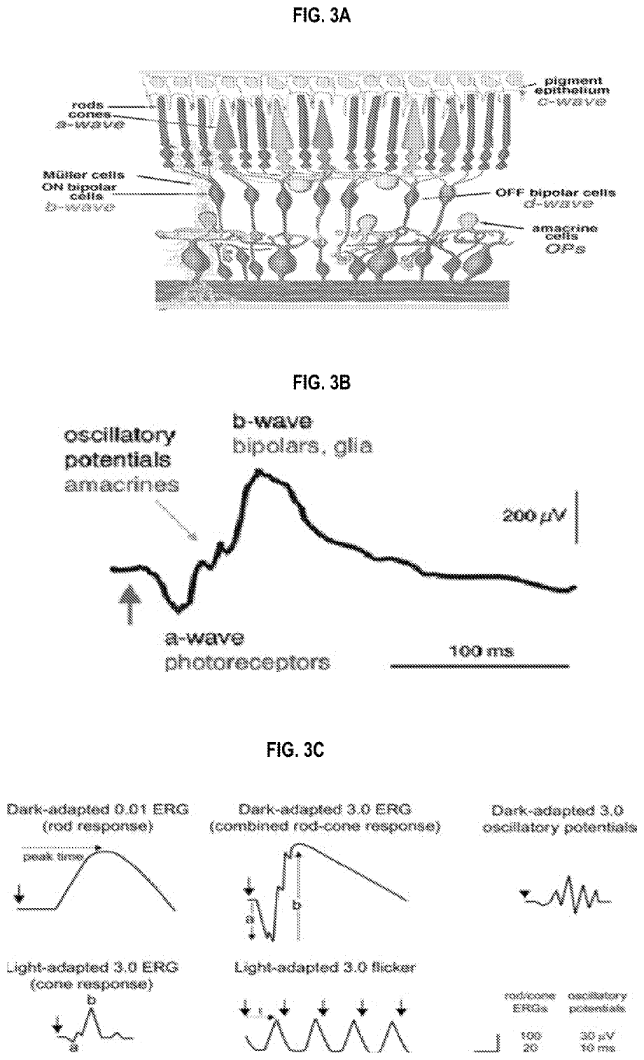

FIG. 3A shows another diagram of the cells of the eye with designations indicating one or more associations between one or more cell types and one or more signals. For example, as seen in FIG. 3A, the one or more rods and cones are associated with waveform features such as one or more a-wave signal, one or more Muller cells (e.g., “On” bipolar cells) are associated with one or more b-wave signals, the pigment epithelium is associated with one or more c-wave signals, one or more “off” bipolar cells are associated with one or more d-wave signals, and the one or more amacrine cells are associated with one or more oscillatory potentials (e.g., “OPs”). FIG. 3B shows an associated ERG signal comprising one or more waves (e.g., wavelets) such as the a-wave associated with photoreceptors like the one or more rods and/or the one or more cones, the OPs associated with the one or more amacrine cells, and the b-wave associated with the one or more bipolar cells and/or glia. FIG. 3C shows a variety of waveforms including a scotopic negative response (STR), which is the retinal ganglion cell response, a photopic waveform (light adapted response), and a flicker waveform (cone response). The methods and systems described herein may determine any waveform features described herein including those features identified in FIGS. 3A-3C.

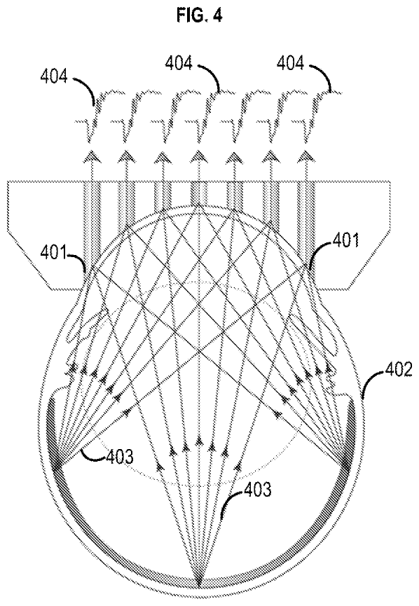

FIG. 4 shows an example diagram of a use case wherein one or more electrodes 401 (e.g., disposed within a contact or other component) are disposed proximate the eye 402. One or more wavelengths of light are directed into the eye wherein they elicit one or more electrochemical responses (e.g., one or more corneal potentials) 403 that may be detected (e.g., determined, measured, processed) by the one or more electrodes 401 and converted into the one or more ERG signals 404. The one or more electrodes may comprise Burian-Allen electrodes (consists of an annular ring of stainless steel surrounding a polymethylmethacrylate (PMMA) contact-lens core. BA electrodes incorporate a lid speculum, which helps to minimize eye blinks/closure. BA lenses are reusable and are available in sizes ranging from pediatric to adult), Dawson-Trick-Litzkow electrodes (low-mass conductive silver/nylon thread, DTL electrodes are disposable and are typically more comfortable for the patients, as compared to other corneal electrodes), Jet electrodes (disposable plastic lens with a gold-plated peripheral circumference), skin electrodes (may be used as a replacement for corneal electrodes by placing an electrode on the skin over the infraorbital ridge near lower eyelid. ERG amplitudes tend to be small and noisy, but skin electrodes are better-tolerated in pediatric populations), mylar electrodes (aluminized or gold-coated Mylar), Cotton-Wick electrodes (Burian-Allen electrode shell fitted with a cotton wick, which is useful for minimizing light-induced artifacts), Hawlina-Konec Electrodes (Teflon-insulated thin metal wire (silver, gold, platinum) with three central windows, 3 mm in length, molded to fit into the lower conjunctival sac), combinations thereof, and the like.

Electrical activity from the corneal electrode is compared to that of a reference electrode placed at a distant site (ear, forehead, temple are common). A differential amplifier is typically used to amplify the difference between two inputs (corneal electrode and reference electrode) and reject signals that are common to both inputs (relative to a ground electrode placed at a third site). Reference and ground electrodes are commonly made of a highly conductive material that is fixed to the patient with paste. Gold cup electrodes are common, because they can be reused; disposable adhesive skin electrodes are also available. Some corneal electrodes contain a reference, which obviates the need for a reference to be placed elsewhere (e.g. BA bipolar electrodes and some skin electrodes).

The full-field ERG is a mass response of the retina that has contributions from several retinal sources, summed throughout the retina. This is useful in diseases that have widespread retinal dysfunction: e.g. rod/cone dystrophies, cancer associated retinopathy, and toxic retinopathies. The ffERG waveform components and their underlying sources depend on both the strength of the stimulus flash and the state of adaptation. That is, scotopic measurements that target rod-pathway function are made from the dark-adapted eye, whereas photopic measurement that target cone-pathway function are made from the light-adapted eye.

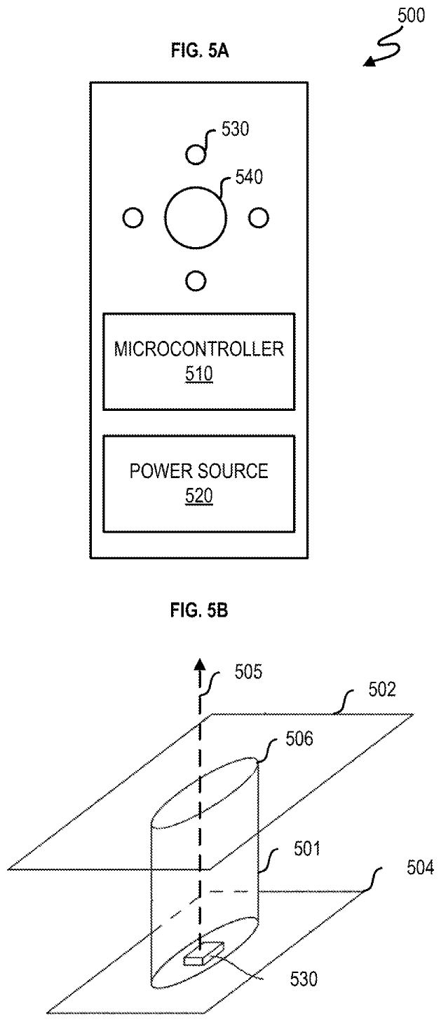

FIG. 5A illustrates a lighting device 500 according to various embodiments of the present disclosure. The lighting device 500 may comprise a microcontroller 510, a power source 520, one or more light sources 530, and one or more light sources 540. In one embodiment, the microcontroller 510 may include and/or be in communication with, an analog emitter source driver, such as an LED driver, to selectively provide power to the one or more light sources 530 and/or the one or more light sources 540. In an embodiment, the one or more light sources 530 may form an LED array. The microcontroller 510 may selectively provide power to the LED array. In one non-limiting example, the analog emitter source driver may include a low noise analog LED driver as one or more adjustable current sources to selectively set and/or adjust (e.g., vary) the lighting parameters (e.g., intensity, frequency, location within field of view, color, pattern, combinations thereof, and the like). The microcontroller 510 may also communicate with a memory, or other onboard storage device configured for storing and reading data.

In one embodiment, the microcontroller 510 may be configured to transmit and/or receive data via a wireless network interface to and/or from an external device (e.g., the ERG device 101). The microcontroller may comprise the wireless network interface. The wireless network interface may be a Bluetooth connection, an antenna, or other suitable interface. In one embodiment, the wireless network interface is a Bluetooth Low Energy (BLE) module. In one non-limiting example, the wireless network interface and the microcontroller 510 are integrated in one unitary component.

The one or more light sources 530 and one or more light sources 540 may comprise one or more LEDs. The one or more light sources 530 may be configured to assist in aligning the lighting device 500 to a user's vision in order to execute the ERG program. The one or more light sources 530 may be recessed within a housing of the lighting device 500. The one or more light sources 540 may be configured to emit light at varying wavelengths, intensities, patterns, frequencies, combinations thereof, and the like in order to execute the ERG program. For example, the lighting device 500 may be configured to administer (e.g., output) one or more light protocols associated with an ERG regimen. For example, the lighting device 500 may be configured to administer a steady-state ERG, a pattern ERG, a focal ERG, a multifocal ERG. The focal ERG (fERG) is used primarily to measure the functional integrity of the central macula and is therefore useful in providing information in diseases limited to the macula.

In addition, the multifocal ERG (discussed below) can be used to assess macular function. The electrode types and placement discussed for the ffERG can also be applied for fERG measurement. A variety of approaches have been described in the literature for recording fERGs. Differing field sizes varying from 3 degrees to 18 degrees and stimulus temporal frequencies have been used in the various methods. However, each technique must address the challenge of limiting amount of light scattered outside the focal test area. fERG is useful for assessing macular function in conditions such as age-related macular degeneration. The multifocal ERG (mfERG) assesses many local ERG responses, typically 61 or 103, within the central 30 degrees. This provides important spatial information that is lacking in the ffERG, allowing dysfunction within the macula that might be missed by ffERG to be assessed. mfERG responses are recorded under light-adapted conditions from the cone-pathway. It is important to note that mfERG is not a replacement for the ffERG: if pan-retinal damage or rod pathway dysfunction is suspected, then the ffERG should also be performed.

The pattern ERG (pERG) uses contrast reversing pattern stimuli (sinewave gratings or checkerboards) to assess macular retinal ganglion cell (RGC) activity. Electrodes and their placement may be the same as those described for the ffERG. However, contact lens electrodes are often avoided to maintain optimal optical quality of the stimulus. Clarity of the ocular media and proper refraction are important for pERG measurement. The pERG is typically recorded with natural pupils. ISCEV has provided a standard for recording the pERG that has most recently been updated in 2012. An example of a common pERG stimulus is shown below (See FIG. 3, left). Over time, the dark checks become light, and the light checks become dark (typically at a rate of 4 reversals per second). It is important that there is no net change in luminance during the dark-to-light transition of the checks (i.e. the average luminance of the screen must be constant over time), or a luminance artifact will be introduced into the response. Given that the pERG responses have relatively small amplitude, many repetitions are obtained in clinical practice.

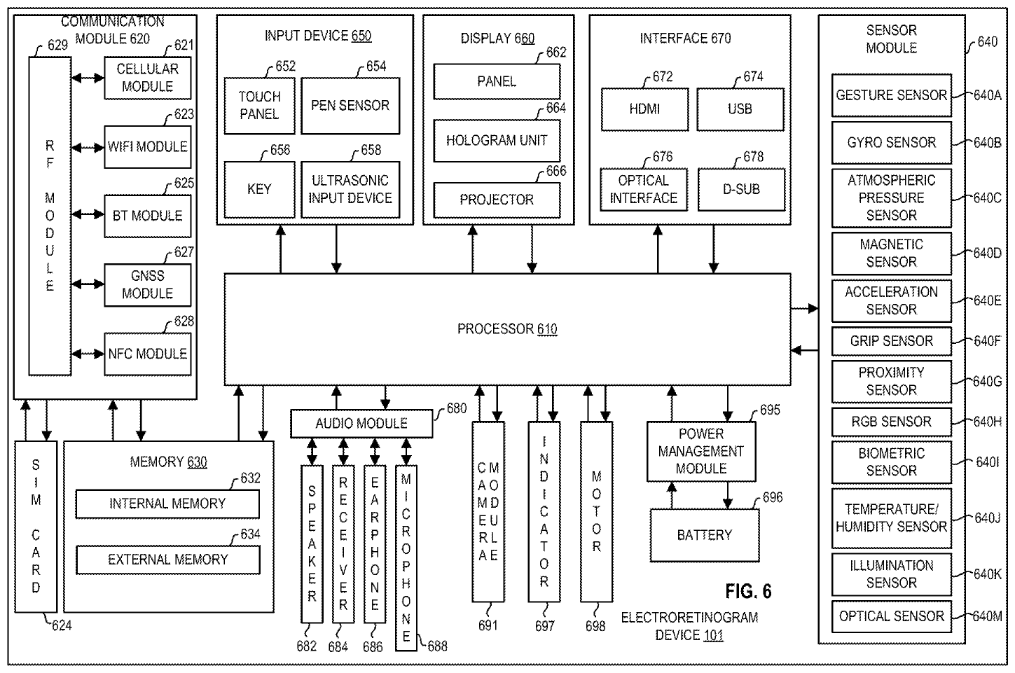

Further, any of the sensors described herein may be used to align the lighting device 500 to a user's vision. For example, a gyro sensor (e.g., gyro sensor 640B as seen in FIG. 6) may determine a vertical or horizontal orientation relative to the ground. Upon determining that the lighting device 500 is orientated approximately perpendicular to the ground, the lighting device 500 may indicate to the user that the lighting device 500 is oriented as such. For example, the one or more light sources 530 may indicate the orientation by, for example, blinking, or changing color or intensity. The lighting device 500 may send a message to the device comprising the user interface element wherein the message indicates the orientation of the lighting device 500. For example, one or more audio tones or visual cues may indicate to the user that the lighting device 500 is properly aligned for user. The one or more light sources 540 may comprise a wide range LED technologies of various luminous intensities.

FIG. 5B shows a simplified perspective view of an illustrative light source recess 501 configured for constraining both vertical and horizontal directions of light emitted from the light source 530 (e.g., so as to minimize ambient light). The light source recess 501 may travel from an exterior housing 502 to an internal mounting surface 504. The light source 530 may be mounted on the internal mounting surface 504. The light source recess 501 may be configured such that light emitted by the light source 530 travels in a specific direction 505 when exiting an opening 506. The direction 505 may be configured to, in conjunction with light existing multiple other openings 506 in the lighting device 500, focus light such that a user of the lighting device 500 will only see all light emitted from all light sources 530 when the lighting device 500 is properly aligned to the user's vision.

FIG. 6 is a block diagram of an electroretinogram (ERG) device 101 according to various exemplary embodiments. The ERG device 101 may include, for example, all or some parts of the ERG device 101, the lighting device 102, or the electrode device 104 of FIG. 1. The ERG device 101 may include one or more processors (e.g., Application Processors (APs)) 610, a communication module 620, a subscriber identity module 624, a memory 630, a sensor module 640, an input device 650, a display 660, an interface 670, an audio module 680, a camera module 691, a power management module 695, a battery 696, an indicator 697, and a motor 698.

The processor 610 may control a plurality of hardware or software constitutional elements connected to the processor 610 by driving, for example, an operating system or an application program, and may process a variety of data, including multimedia data and may perform an arithmetic operation. The processor 610 may be implemented, for example, with a System on Chip (SoC). According to one exemplary embodiment, the processor 610 may further include a Graphic Processing Unit (GPU) and/or an Image Signal Processor (ISP). The processor 610 may include at least one part (e.g., a cellular module 621) of the aforementioned constitutional elements of FIG. 1. The processor 610 may process an instruction or data, which is received from at least one of different constitutional elements (e.g., a non-volatile memory), by loading it to a volatile memory and may store a variety of data in the non-volatile memory.

The communication module 620 may have a structure the same as or similar to the communication interface 170 of FIG. 1. The communication module 620 may include, for example, the cellular module 621, a Wi-Fi module 623, a Bluetooth (BT) module 625, a GNSS module 627 (e.g., a GPS module, a Glonass module, a Beidou module, or a Galileo module), a Near Field Communication (NFC) module 628, and a Radio Frequency (RF) module 629.

The cellular module 621 may provide a voice call, a video call, a text service, an internet service, or the like, for example, through a communication network. According to one exemplary embodiment, the cellular module 621 may identify and authenticate the ERG device 101 in the communication network by using the subscriber identity module (e.g., a Subscriber Identity Module (SIM) card) 624. According to one exemplary embodiment, the cellular module 621 may perform at least some functions that can be provided by the processor 610. According to one exemplary embodiment, the cellular module 621 may include a Communication Processor (CP).

Each of the WiFi module 623, the BT module 625, the GNSS module 627, or the NFC module 628 may include, for example, a processor for processing data transmitted/received via a corresponding module. According to a certain exemplary embodiment, at least one of the cellular module 621, the WiFi module 623, the BT module 625, the GPS module 627, and the NFC module 628 may be included in one Integrated Chip (IC) or IC package.

The RF module 629 may transmit/receive, for example, a communication signal (e.g., a Radio Frequency (RF) signal). The RF module 629 may include, for example, a transceiver, a Power Amp Module (PAM), a frequency filter, a Low Noise Amplifier (LNA), an antenna, or the like. According to another exemplary embodiment, at least one of the cellular module 621, the WiFi module 623, the BT module 625, the GPS module 627, and the NFC module 628 may transmit/receive an RF signal via a separate RF module.

The subscriber identity module 624 may include, for example, a card including the subscriber identity module and/or an embedded SIM, and may include unique identification information (e.g., an Integrated Circuit Card Identifier (ICCID)) or subscriber information (e.g., an International Mobile Subscriber Identity (IMSI)).

The memory 630 (e.g., the memory 130) may include, for example, an internal memory 632 or an external memory 634. The internal memory 632 may include, for example, at least one of a volatile memory (e.g., a Dynamic RAM (DRAM), a Static RAM (SRAM), a Synchronous Dynamic RAM (SDRAM), etc.) and a non-volatile memory (e.g., a One Time Programmable ROM (OTPROM), a Programmable ROM (PROM), an Erasable and Programmable ROM (EPROM), an Electrically Erasable and Programmable ROM (EEPROM), a mask ROM, a flash ROM, a flash memory (e.g., a NAND flash memory, a NOR flash memory, etc.), a hard drive, or a Solid State Drive (SSD)).

The external memory 634 may further include a flash drive, for example, Compact Flash (CF), Secure Digital (SD), Micro Secure Digital (Micro-SD), Mini Secure digital (Mini-SD), extreme Digital (xD), memory stick, or the like. The external memory 634 may be operatively and/or physically connected to the ERG device 101 via various interfaces.

The sensor module 640 may measure, for example, a physical quantity or detect an operational status of the ERG device 101, and may convert the measured or detected information into an electric signal. The sensor module 640 may include, for example, at least one of a gesture sensor 640A, a gyro sensor 640B, a pressure sensor 640C, a magnetic sensor 640D, an acceleration sensor 640E, a grip sensor 640F, a proximity sensor 640G, a color sensor 640H (e.g., a Red, Green, Blue (RGB) sensor), a biometric sensor 640I, a temperature/humidity sensor 640J, an illumination sensor 640K, an optical sensor 640M. According to one exemplary embodiment, the optical sensor 640M may detect ambient light and/or light reflected by an external object (e.g., a user's finger. etc.), and convert the detected ambient light into a specific wavelength band by means of a light converting member. For example, the illumination sensor 640K may comprise a light meter sensor. An exemplary sensor may be the Amprobe LM-200LED, however any suitable light meter sensor may be used. In an embodiment, the illumination sensor 640K may be pressed against a diffuser of the lighting device. Additionally or alternatively, the sensor module 640 may include, for example, an E-nose sensor, an ElectroMyoGraphy (EMG) sensor, an ElectroEncephaloGram (EEG) sensor, an ElectroCardioGram (ECG) sensor, an Infrared (IR) sensor, an iris sensor, and/or a fingerprint sensor. The sensor module 640 may further include a control circuit for controlling at least one or more sensors included therein. In a certain exemplary embodiment, the ERG device 101 may further include a processor configured to control the sensor module 604 either separately or as one part of the processor 610, and may control the sensor module 640 while the processor 610 is in a sleep state.

The input device 650 may include, for example, a touch panel 652, a (digital) pen sensor 654, a key 656, or an ultrasonic input device 658. The touch panel 652 may recognize a touch input, for example, by using at least one of an electrostatic type, a pressure-sensitive type, and an ultrasonic type detector. In addition, the touch panel 652 may further include a control circuit. The touch panel 652 may further include a tactile layer and thus may provide the user with a tactile reaction (e.g., haptic feedback). For instance, the haptic feedback may be associated with the executing the ERG program. The haptic feedback may be associated with the user input.

The (digital) pen sensor 654 may be, for example, one part of a touch panel, or may include an additional sheet for recognition. The key 656 may be, for example, a physical button, an optical key, a keypad, or a touch key. The ultrasonic input device 658 may detect an ultrasonic wave generated from an input means through a microphone (e.g., a microphone 688) to confirm data corresponding to the detected ultrasonic wave.

The display 660 (e.g., the display 160) may include a panel 662, a hologram unit 664, or a projector 666. The panel 662 may include a structure the same as or similar to the display 160 of FIG. 1. The panel 662 may be implemented, for example, in a flexible, transparent, or wearable manner. The panel 662 may be constructed as one module with the touch panel 652. According to one exemplary embodiment, the panel 662 may include a pressure sensor (or a force sensor) capable of measuring a pressure of a user's touch. The pressure sensor may be implemented in an integral form with respect to the touch panel 652, or may be implemented as one or more sensors separated from the touch panel 652.

The hologram unit 664 may use an interference of light and show a stereoscopic image in the air. The projector 666 may display an image by projecting a light beam onto a screen. The screen may be located, for example, inside or outside the ERG device 101. According to one exemplary embodiment, the display 660 may further include a control circuit for controlling the panel 662, the hologram unit 664, or the projector 666.

The interface 670 may include, for example, a High-Definition Multimedia Interface (HDMI) 672, a Universal Serial Bus (USB) 674, an optical communication interface 676, or a D-subminiature (D-sub) 678. The interface 670 may be included, for example, in the communication interface 170 of FIG. 1. Additionally or alternatively, the interface 670 may include, for example, a Mobile High-definition Link (MHL) interface, a Secure Digital (SD)/Multi-Media Card (MMC) interface, or an Infrared Data Association (IrDA) standard interface.

The audio module 680 may bilaterally convert, for example, a sound and electric signal. At least some constitutional elements of the audio module 680 may be included in, for example, the input/output interface 150 of FIG. 1. The audio module 680 may convert sound information, which is input or output, for example, through a speaker 682, a receiver 684, an earphone 686, the microphone 688, or the like.

The camera module 691 may comprise, for example, a device for image and video capturing, and according to one exemplary embodiment, may include one or more image sensors (e.g., a front sensor or a rear sensor), a lens, an Image Signal Processor (ISP), or a flash (e.g., LED or xenon lamp).

The power management module 695 may manage, for example, power (e.g., consumption or output) of the ERG device 101. According to one exemplary embodiment, the power management module 695 may include a Power Management Integrated Circuit (PMIC), a charger Integrated Circuit (IC), or a battery fuel gauge. The PMIC may have a wired and/or wireless charging type. The wireless charging type may include, for example, a magnetic resonance type, a magnetic induction type, an electromagnetic type, or the like, and may further include an additional circuit for wireless charging, for example, a coil loop, a resonant circuit, a rectifier, or the like. A battery gauge may measure, for example, residual quantity of the battery 696 and voltage, current, and temperature during charging. The battery 696 may include, for example, a non-rechargeable battery, a rechargeable battery, and/or a solar battery.

The indicator 697 may display a specific state, for example, a booting state, a message state, a charging state, or the like, of the ERG device 101 or one part thereof (e.g., the processor 610). The motor 698 may convert an electric signal into a mechanical vibration, and may generate a vibration or haptic effect. Although not shown, the ERG device 101 may include a processing device (e.g., a GPU) for supporting a mobile TV. The processing device for supporting the mobile TV may process media data conforming to a protocol of, for example, Digital Multimedia Broadcasting (DMB), Digital Video Broadcasting (DVB), MediaFlo™, or the like.

Each of the constitutional elements described in the present document may consist of one or more components, and names thereof may vary depending on a type of an electronic device. The electronic device, according to various exemplary embodiments, may include at least one of the constitutional elements described in the present document. Some of the constitutional elements may be omitted, or additional other constitutional elements may be further included. Further, some of the constitutional elements of the electronic device, according to various exemplary embodiments, may be combined and constructed as one entity so as to equally perform functions of corresponding constitutional elements before combination.

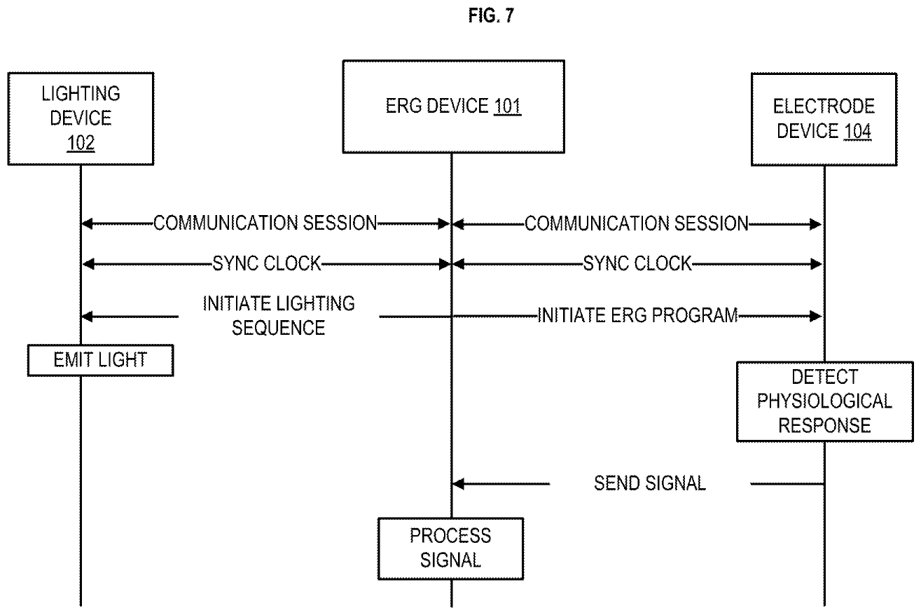

FIG. 7 illustrates an ERG program process according to various embodiments of the present disclosure. The ERG device 101 may open a communication session with the lighting device 102 and electrode device 104. Optionally, the ERG device 101 may send an instruction to the electrode device 104 to synchronize internal clocks of both devices.

Similarly, the ERG device 101 may send an instruction to the lighting device 102 to synchronize internal clocks of both devices. The ERG device 101 may send an instruction to the lighting device 102 to cause the lighting device 102 to initiate a lighting sequence. The instruction may comprise the one or more lighting parameters. The lighting device 102 may comprise one or more light sources (e.g., the one or more light sources 530 and the one or more light sources 540). In various embodiments, the instruction may cause light to be emitted from, for example, one or more of the one or more light sources 530 and/or the one or more light sources 540. The one or more light sources may comprise one or more LEDs. The one or more light sources 530 may be configured to assist in aligning the lighting device 102 to a user's vision in order to execute the ERG program. The one or more light sources 530 may be recessed within a housing of the lighting device 500. The one or more light sources 540 may be configured to emit light at varying wavelengths, intensities, patterns, frequencies, combinations thereof, and the like in order to execute the ERG program. For example, the lighting device 500 may be configured to administer (e.g., output) one or more light protocols associated with an ERG regimen. For example, the lighting device 102 may be configured to administer a steady-state ERG, a pattern ERG, a focal ERG, a multifocal ERG, combinations thereof and the like as described herein. According to various embodiments, the ERG device 101 may initiate an ERG program by communicating with the lighting device 102 to cause the lighting device 102 to emit light. The ERG device 101 may cause the lighting device 102 to vary the one or more lighting parameters. The emitted light, upon being viewed by a subject (e.g., a human or animal) may elicit a physiological response (e.g., an electrochemical signal) in the eye of the subject.

The ERG device 101 may send an instruction to the electrode device 104 to cause the electrode device 104 to initiate an ERG measurement process. The electrode device 104 may receive the instruction. The electrode device 104 may detect a physiological response signal. The electrode device 104 may detect the electrochemical signal (e.g., one or more electrical responses associated with one or more cell types). The electrode device 104 may relay the signal to the ERG device 101. The ERG device may process the signal as described further herein. The ERG device 101 may repeat the process and log the results. In various embodiments, the ERG device 101 may cause the lighting device 102 to emit light at a first intensity and increase or decrease the intensity. In various embodiments, the ERG device 101 may cause the lighting device 102 to emit light of a first pattern (e.g., points of light, a checkerboard, circles of light, other shapes or patterns, combinations thereof, and the like) and change the first pattern to a second pattern. In various embodiments, the ERG device 101 may cause the lighting device 102 to emit light at a first color and change the color to a second color. In various embodiments, the ERG device 101 may cause the lighting device 102 to emit light at a first frequency and increase or decrease the first frequency to a second frequency.

According to various embodiments, the electrode device 104 may transmit data indicative of a physiological response signal to the ERG device 101 (e.g., a remote server). According to various embodiments, the electrode device 104 may be connected to the ERG device 101 through wireless communication and may receive data from the ERG device 101 in real time. According to various embodiments, the electrode device 104 may display various User Interfaces (UIs) or Graphical User Interfaces (GUIs) based at least partially on the received data.

According to various embodiments, the ERG device 101 may include, for example, a smartphone, tablet, Personal Digital Assistant (PDA), a tablet, a Personal Computer (PC), combinations thereof, and the like. According to various embodiments, the ERG device 101 may display various UIs or GUIs related to using the lighting device 102. The operation and relevant screen examples of the ERG device 101 according to various embodiments will be described in detail with reference to the figures below.

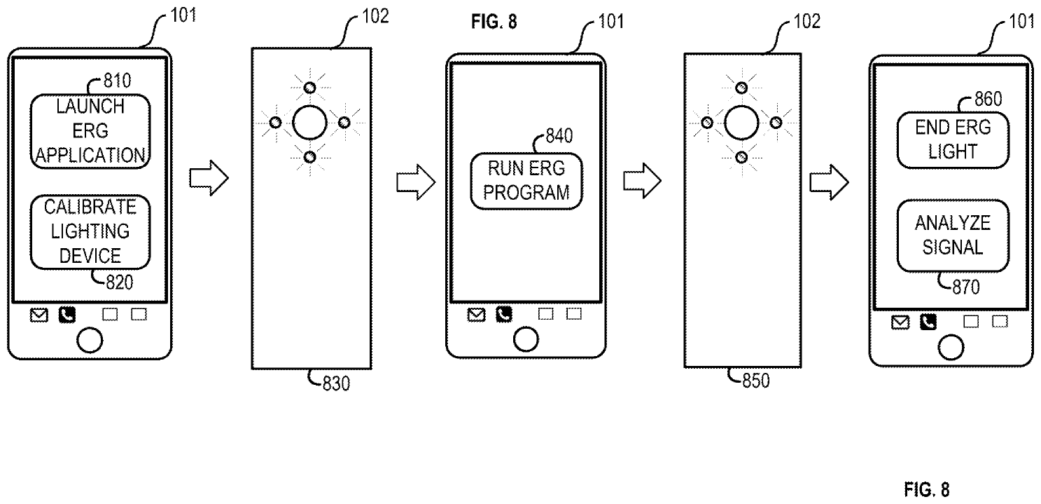

FIG. 8 illustrates an ERG lighting method according to various embodiments of the present disclosure. While the ERG device 101 is depicted as a user device (e.g., a smartphone), it is to be understood that the ERG device 101, as described herein, may be any computing device (including the computer 1101 or any of the remote computing devices 1114A-C described herein. For example, at step 810, a user may user may launch an ERG application (e.g., software program) resident on the ERG device 101. For example, launching the ERG application may comprise initializing the ERG application and opening one or more communication session with one or more other devices. For example, the ERG application may initiate a communication session with the electrode device 104 and/or the lighting device 102. At step 820, the lighting device 102 may be calibrated. Calibrating the lighting device 102 may comprise establishing baseline lighting parameters and ensuring the lighting device 102 is functioning properly. At step 830, the lighting device may be caused to output a calibrating light output. The user may engage a user interface element on the ERG device 101 to calibrate the electrode device 104. In response, the lighting device 102 may activate one or more light sources on the lighting device 102. For example, the one or more light sources may be recessed (as described above) such that the user may only view the light when the recess is level with the eyes of the user (e.g., the viewing angle is around 0 degrees). For example, the lighting device may be caused to emit a first light comprising a given lighting parameter such as an intensity (e.g., luminance), color, frequency, wavelength, etc. and the output may be confirmed. At step 840, an ERG program may be run. Running the ERG program may comprise causing an ERG light regimen to be output. For example, the lighting device 102 may be configured to administer a steady-state ERG, a pattern ERG, a focal ERG, a multifocal ERG, combinations thereof and the like as described herein. According to various embodiments, the ERG device 101 may initiate an ERG program by communicating with the lighting device 102 to cause the lighting device 102 to emit light. The ERG device 101 may cause the lighting device 102 to vary the one or more lighting parameters.

The user may engage the user interface element on the ERG device 101 to start an ERG measurement process (e.g., ERG Test). The electrode device 104 may detect a physiological response (e.g., an electrochemical signal). For example, the electrode device 104 may be configured to determine (e.g, detect, measure, receive) one or more corneal potentials. The one or more corneal potentials may be associated with, an ERG signal. The one or more corneal potentials, and the ERG signals (and/or components such as “waves” thereof) may be associated with one or more parts of the eye. For example, one or more wavelengths of light are directed into the eye wherein they elicit one or more electrochemical responses (e.g., one or more corneal potentials) that may be detected (e.g., determined, measured, processed) by the one or more electrodes and converted into the one or more ERG signals.

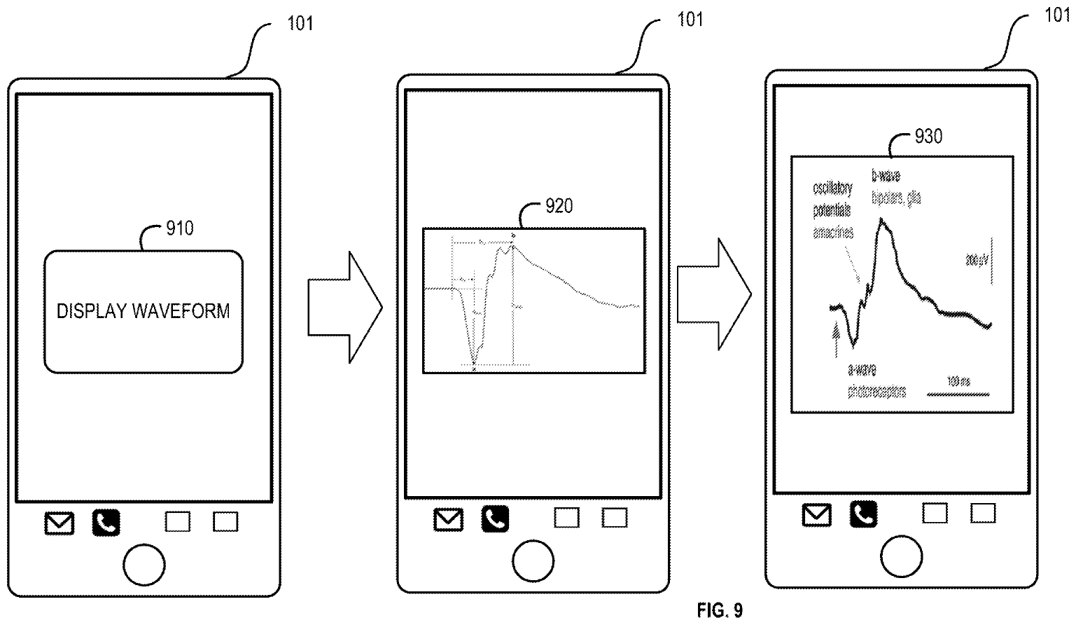

The electrode device 104 may relay the electrochemical signal to the ERG device 101. At step 860, the user may terminate the ERG light regiment. At 870, the user may engage the user interface element to initiate a signal analysis as described in greater detail with respect to FIGS. 9-11. For example, electrical activity from the corneal electrode is compared to that of a reference electrode placed at a distant site (ear, forehead, temple are common). A differential amplifier is typically used to amplify the difference between two inputs (corneal electrode and reference electrode) and reject signals that are common to both inputs (relative to a ground electrode placed at a third site). In an embodiment, the ERG device 101 may guide the user through the ERG signal analysis process. In an embodiment, the ERG device 101 may process the signal automatically as described further herein.

FIG. 9 shows an example method 900 as implemented on any one or more of the devices described herein, such as, for example, the ERG device 101. At step 910, a user may input a user input via a user interface associated with the ERG device 101. The user input may be received by the user interface. For example, the user interface may comprise a touchscreen, or any other suitable user interface. For example, the user interface may display a selectable option. The selectable option may be configured to receive the user input. The selectable option may be configured to cause, based on the received user input, a display of one or more waveforms. The one or more waveforms may comprise a raw waveform, a denoised waveform, a filtered waveform, or any other waveform described herein. At steps 920 and/or 930, a user may manually identify one or more features of the one or more waveforms. For example, the user may mark (e.g., via a finger, a stylus, a cursor, or any other method), one or more peaks, one or more troughs, one or more amplitudes, one or more periods, or any other features. For example, as seen at 920, a user has identified one or more peaks, one or more troughs, and one or more amplitudes between the one or more peaks and the one or more troughs. For example, as seen at 930, a user identified particular signal features (e.g., one or more oscillatory potentials, an a-wave and a b-wave).

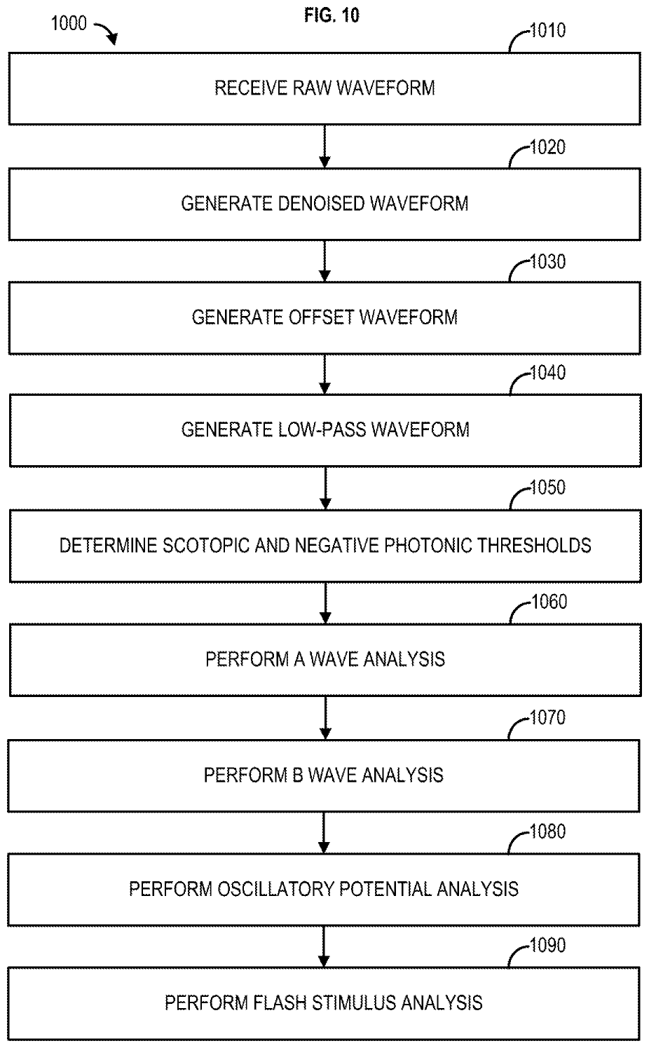

FIG. 10 shows an example method 1000. The method 1000 may be implemented by any suitable computing device such as the ERG device 101, the lighting device 102, the electrode device 104, combinations thereof, and/or any other devices described herein. The method may be predicated on initiating an ERG program. Initiating the ERG program may comprise initiating an ERG application (e.g., a software application) comprising a user interface. The software application may comprise a user interface. A user may interact with the ERG application. Via the ERG application the user may establish ERG program settings. For example, whether or not an ERG analysis will take advantage of flash intensity information may be defined. Flash intensity information may relate to the intensity of the emitted light (e.g., luminance as measured, for example in candles per square meter or “cd/m²”). If not, the ERG analysis will not take into account the flash intensity information. That is to say, if flash intensity is ignored, all waves will be analyzed based on the waves' individual properties. For example, if flash intensity is ignored, scotopic threshold response (STR) and negative photopic response (nPHR) may not be assessed automatically, but peaks may be marked as identified features of a wave. However, all features may be manually placed if desired. Additionally, if a flash stimulus is introduced and flash intensity is ignored, a received waveform associated with a physiological response (e.g., a raw waveform) will be analyzed as if the raw waveform were a normal flash response. However, if flash intensity is accounted for, flash intensity parameters may be determined. For example, a number of steps (e.g., a number of ERG flashes per subject), flash intensity (in cd/m² or log cd/m²), and lighting condition (e.g., scotopic or dark adapted, photopic [light adapted] or normal, and flicker), a sampling frequency associated with the waveform, a flicker frequency (e.g., as measured in Hz), flash time (e.g., duration of flash in seconds, time between flashes in seconds, combinations thereof, and the like). A filter type may be defined. For example, the ERG analysis may implement a Butterworth filter, a finite impulse response filter (FIR), a lowpass filter, a highpass filter, a bandpass filter, a notch filter, combinations thereof, and the like. The Butterworth filter is a type of signal processing filter designed to have a frequency response that is as flat as possible in the passband. It is also referred to as a maximally flat magnitude filter. The frequency response of the Butterworth filter is maximally flat (i.e. has no ripples) in the passband and rolls off towards zero in the stopband. When viewed on a logarithmic Bode plot, the response slopes off linearly towards negative infinity. A first-order filter's response rolls off at −6 dB per octave (−20 dB per decade) (all first-order lowpass filters have the same normalized frequency response). A second-order filter decreases at −12 dB per octave, a third-order at −18 dB and so on. Butterworth filters have a monotonically changing magnitude function with co, unlike other filter types that have non-monotonic ripple in the passband and/or the stopband.

A finite impulse response (FIR) filter is a filter whose impulse response (or response to any finite length input) is of finite duration, because it settles to zero in finite time. The impulse response (that is, the output in response to a Kronecker delta input) of an Nth-order discrete-time FIR filter lasts exactly N+1 samples (from first nonzero element through last nonzero element) before it then settles to zero. FIR filters can be discrete-time or continuous-time, and digital or analog.

At 1010 a raw waveform may be received. In executing the ERG program, a stimulus may be introduced to a patient and a physiological response received. For example, the electrode device 104 may be configured to determine (e.g, detect, measure, receive) one or more corneal potentials. The one or more corneal potentials may be associated with, an ERG signal. The one or more corneal potentials, and the ERG signals (and/or components such as “waves” thereof) may be associated with one or more parts of the eye. For example, one or more wavelengths of light are directed into the eye wherein they elicit one or more electrochemical responses (e.g., one or more corneal potentials) that may be detected (e.g., determined, measured, processed) by the one or more electrodes and converted into the one or more ERG signals. The physiological response may be associated with a raw waveform. The raw waveform may comprise a signal comprising voltage data, current data, timing data, frequency data, combinations thereof, and the like. The raw waveform may be displayed on the display.

At 1020 a denoised waveform may be determined. The raw waveform may undergo a denoising process and wavelet analysis. The denoising process may comprise using 1-D wavelet denoising which may use the maximal overlap discrete wavelet transformation. The denoising process may use local scaling to reduce artificial noise in the raw waveform while maintaining natural oscillations and/or peaks of the raw waveform. A base level of variation in the raw waveform can be determined. The base level of variation may be determined based on the denoised waveform. The base level of variation may be between 20% and 40% of a pre-flash recording. The pre-flash recording may comprise a time where the electrical signal and time are recorded prior to initiating flash (e.g., the light stimulus). This time typically encompasses the noise and variation inherent in the waveform signal. However, if the pre-flash is absent, a baseline parameter may be determined based on the first 0.25 ms of the flash stimulus or later defined manually by the user. A region of the denoised waveform may be determined. The region of the denoised waveform may comprise an area (e.g., as measured in units of time and voltage) of the denoised waveform. A confidence interval of the recorded voltage may be determined. The confidence interval may be associated with a signal variation in the determined area of the denoised waveform. The confidence interval may be associated with a lower and upper variation threshold as an estimate of the noise of the recorded voltage.

At 1030 an offset waveform may be determined. Determining the offset waveform may comprise determining an average (e.g., mean). The mean may be associated with a region of the raw waveform or the denoised waveform. Determining the offset waveform may be used to adjust the denoised waveform by a signal offset. This offset waveform may translate the raw waveform or denoised waveform so that the denoised region is centered at zero volts.

At 1040 a low pass waveform may be determined. Determining the lowpass waveform may comprise applying a lowpass filter to the offset waveform. For example, the lowpass filter may comprise a lowpass zero-phase digital filter. For example, determining the lowpass waveform may comprise applying a 5^{th} order Butterworth filter with a low frequency cutoff of around 60 Hz to the offset waveform. The order of the Butterworth filter may be adjusted. If desired, the user may also adjust the filter type, for example from a Butterworth filter to an alternative digital filter.

At 1050, a scotopic threshold response (STR) and negative photopic threshold (NPT) analysis may be performed. For example, if flash intensities were defined and the flash intensity is below −4 log cd*s/m2. The positive STR (pSTR) may be defined as the maximum amplitude and implicit time of this lowpass waveform. The pSTR implicit time may then be used to define the location of the pSTR on the denoised waveform to identify this signal. If flash intensity was defined, the minimum amplitude of the lowpass waveform may be found. For example, if the flash intensity was defined and was less than −4 log cd*s/m2 in a dark adapted or scotopic step, the location of the minimum amplitude may define the negative STR (nSTR) on the lowpass filtered waveform. The aforementioned flash intensity is merely exemplary and a person skilled in the art will appreciate that the flash intensity and thresholds associated therewith may very among and between devices. The implicit time of the nSTR may be used to define the amplitude and implicit time of the nSTR on the offset waveform. If the flash intensities are defined and it is a photopic step the method may also identify the minimum amplitude between the b-wave amplitude and the end of the signal as the negative photopic threshold.

At 1060, an a-wave analysis may be performed if flash intensities are not defined and/or are above the defined flash intensities for the scotopic threshold. If the flash intensities are not defined, as dark adapted or scotopic, above the STR range, or photopic the negative amplitude of the Lowpass Waveform must pass four thresholds 1) The amplitude must be two times the Lower Variation Threshold, 2) The amplitude must have a magnitude of at least 5 microvolts, 3) this negative amplitude does not occur within 1 ms of the flash stimulus, and 4) the slope of the voltage-time curve from the point of the flash stimulus (˜0 ms) to this minimum. The slope must be greater than 0.5 uV/ms to continue assessing the a-wave. Otherwise the slope may indicate a drift in the signal or oscillations in the response. If the lowpass waveform passes these thresholds the minimums in the Offset Waveform within 10 ms of the minimum of the lowpass Waveform may be determined. These local minimums must be above 2 to 5 microvolts in absolute magnitude and have a minimum peak prominence (local amplitude) of at least 3 microvolts. Performing the a-wave analysis may comprise determining the minimum amplitude of the lowpass waveform. If more than one qualifying local minimum is found several additional conditions may be determined so as to determine the correct location of the a-wave. For example: 1) if more than 1 peak is found a first assumption that no peaks can occur within 2.5 ms of the flash stimulus may be implemented, 2) if flash intensities are used, previous curve information may be included in the analysis, if an a-wave was found on a previous curve it may be assumed the a-wave of an increased flash intensity is faster, thus the next peak that is faster than the previous waveform may be identified, 3) if no flash intensity or previous waveform information is available, the median waveform or first peak after 7 ms may be flagged for the inspection for the user. The array index of the minimum may be used to calculate the a-wave amplitude and implicit time. If multiple a-waves are detected the signal may be flagged for inspection by the user.

At 1070 a b-wave analysis may be performed. If the flash intensities were ignored or were above −4 log cd*s/m2, the maximum amplitude of the lowpass waveform must meet two thresholds: 1) the amplitude must be at least two times the 99% confidence interval of the Upper Variation Threshold, and 2) the amplitude must be greater than 2 microvolts. If these thresholds are not satisfied, the b-wave is not automatically marked. However, if the thresholds are satisfied, the lowpass waveform may be fit with a function, for example,

$$V ( t ) = R P 2 * ( {( ( \frac{c}{( d + 1 )} ) * ( t - {T}_{m} ) + c )}^{( d + 1 )} * {e}^{( t - {T}_{m} ) * \frac{{( d + 1 )}^{2}}{( d + 1 ) * ( t - {T}_{m} ) + c}} )$$

where c represents shape parameters of the lowpass waveform (e.g., of the PII response) and where RP2 is the amplitude of the PII response and Tₘ is the estimated time of the peak of the PII response. The area under the fitted curve is calculated as the energy of the PII response.

If the maximum amplitude passes the described thresholds the b-wave analysis may comprise determining the maximum amplitude of the lowpass waveform. The array index of this maximum amplitude on the lowpass waveform is then determined on the offset waveform and may be used to define the raw amplitude and implicit time of the b-wave. The amplitude of the b-wave may be defined as the amplitude from baseline of the offset wave to the peak of the b-wave or from the amplitude of the a-wave to the peak of the b-wave (trough-to-peak amplitude). For example, if flash intensities were defined and the flash intensity is below −4 log cd*s/m2 the analysis may be based on the STR as described above. The positive STR (pSTR) may be defined as the amplitude and implicit time of this maximum waveform. If flash intensity was defined, the minimum amplitude of the lowpass waveform may be found. For example, if the flash intensity was defined and was less than −4 log cd*s/m2 in a scotopic step, the location of the minimum amplitude may define the negative STR. If the flash intensities are defined and it is a photopic step the method may also identify the minimum amplitude between the b-wave amplitude and the end of the signal as the negative photopic threshold. If the flash intensities are not defined, scotopic but above the STR range, or photopic the negative amplitude of the Lowpass Waveform must pass four thresholds 1) The amplitude must by two times the Lower Variation Threshold, 2) The amplitude must have a magnitude of at least 5 microvolts, 3) this negative amplitude does not occur within 1 ms of the flash stimulus, and 4) the slope of the voltage-time curve from the point of the flash stimulus (˜0 ms) to this minimum. The slope must be greater than 0.5 uV/ms to continue assessing the a-wave. Otherwise the slope may indicate drift in the signal or oscillations in the response. If the lowpass waveform passes these thresholds the Offset Waveform within 10 ms of the minimum of the lowpass Waveform may be determined. These local minimums must be above 2 to 5 microvolts in absolute magnitude and have a minimum peak prominence (local amplitude) of at least 3 microvolts

If more than one qualifying local maximum is found, several additional conditions may be determined so as to determine the correct location of the a-wave. For example: 1) if more than 1 peak is found it may be assumed that no peaks can occur within 2.5 ms of the flash stimulus, 2) if flash intensities are used, previous curve information may be incorporated, if an a-wave was found on a previous curve it may be assumed that the a-wave of an increased flash intensity is faster, and thus the next peak that is faster than the previous waveform may be determined, 3) if no flash intensity or previous waveform information is available, the median waveform or first peak after 7 ms may be marked for the inspection for the user. The array index of the minimum may be used to calculate the a-wave amplitude and implicit time.

However, if the flash intensities were ignored or were above −4 log cd*s/m2, the maximum amplitude of the lowpass waveform must meet two thresholds: 1) the amplitude must be at least two times the 99% CI of the Upper Variation Threshold, and 2) the amplitude must be greater than 2 microvolts. If these threshold are not satisfied, the b-wave is not automatically marked. However, if the thresholds are satisfied, the lowpass waveform may be fit with a function, for example,

$$V ( t ) = R P 2 * ( {( ( \frac{c}{( d + 1 )} ) * ( t - {T}_{m} ) + c )}^{( d + 1 )} * {e}^{( t - {T}_{m} ) * \frac{{( d + 1 )}^{2}}{( d + 1 ) * ( t - {T}_{m} ) + c}} )$$

where c represents shape parameters of the lowpass waveform (e.g., of the PH response) and where RP2 is the amplitude of the PH response and Tₘ is the estimated time of the peak of the PH response. The area under the fitted curve is calculated as the energy of the PH response.

At 1080 an oscillatory potential (OP) analysis may be performed. After assessing the a-wave and b-wave, the offset waveform may be passed through a bandpass filter to generate the OP Waveform. The bandpass filter may be set to 60-235 Hz; however, the frequency and type of filter can be altered by the user as described above. The 99% confidence interval of the tail end of the OP Waveform may be used to determine variation in signal. The tail end of the signal may be used to avoid artificial noise from the flash stimulus or a-wave. To automatically detect OPs the amplitude of the OP Waveform must pass two thresholds: 1) signal peak must by greater than 5 microvolts, and 2) signal peak must be five times the 99% confidence interval. The OP waveform may be normalized to its peak amplitude. A local minimum (trough) and a local maximum (peak) may be determined. The local minimum and local maximum may be determined based on limiting a range to detect OPs based on one or more conditions: 1) if an a-wave is detected, the first minimum within a +1 ms window of the a-wave implicit time is used to find the minimum of the lowpass filtered wave. All OP troughs and peaks below this location are omitted, 2) if no a-wave is detected, the lowpass waveform is again fit to a function as described herein. The second derivative of this function may be found. The peak of the second derivative may be identified. This second peak may be considered the inflection point, or where the leading edge of the b-wave begins. All OP troughs and peaks that occur before a 5 ms window of this inflection point may be omitted. The program may mark OP1-OP5. To confirm the OPS, a normalized OP signal may be scaled by 0.25, 0.5, 1.25 and 1.5. The OP process may be repeated and, if the same OPs are not found the signal may be flagged for manual inspection by the user. Otherwise the OP locations are accepted.

At 1090 a flash stimulus analysis may be performed. A flash stimuli at a specific flash intensity may be defined and then an analysis of the flash stimulus may be performed. If flash intensities are ignored, all flicker steps will be analyzed as a standard ERG waveform. However, these markings can be adjusted by the user manually if desired. Performing the flash stimulus analysis may comprise passing a flicker waveform through a wavelet denoising algorithm as described above. The flicker waveform may then be passed through the lowpass filter. The flicker waveform may be used to find the local troughs and peaks with a minimum amplitude of 8 microvolts spaced based on the flicker stimulus. For example, in a protocol with the stimuli occurring 50 ms apart the minimum space between these local troughs may be at least 50 ms apart. This time interval may be set by the flash stimulus interval and can be modified for quicker or slower flicker stimulus. The first peak may be omitted. The second trough-to-peak value may be used for analysis.

This process may be repeated for each flash intensity and each subject (e.g., patient, animal). There currently is no limit on the number of flash intensities. After all subjects are analyzed the user (e.g., clinician, doctor, medical imaging technician or other user) may be notified and may manually inspect all waveforms. The user has the ability to manually adjust all markings including a-wave, b-wave, OPs, nSTR, pSTR, photopic negative response, and flicker analysis. The user can also automatically mark OPs from a different position. The user can also adjust the baseline position. The user can flag and comment on any waveform. Using the GUI the user can also inspect the raw waveform. After the user is completed with the markings. The user can save progress of the session or the entire session to view later. Afterwards the user can export the data. The exported file may comprise a list of the ID, date tested, flags and user comments for each waveform. The default exported data may also include (if applicable) the a-wave amplitude and implicit time. The a-wave is measured from baseline (either 0 as described above as manually defined for the individual wave by the user) to the trough. Regarding the b-wave amplitude and implicit time, the b-wave is defined as the amplitude from the a-wave to the b-wave index. If there is no a-wave the b-wave amplitude is defined from baseline (either 0 or described above as manually defined by the user) to the peak. The OP amplitude may be defined as the trough-to-peak and the implicit time as the time of each peak.

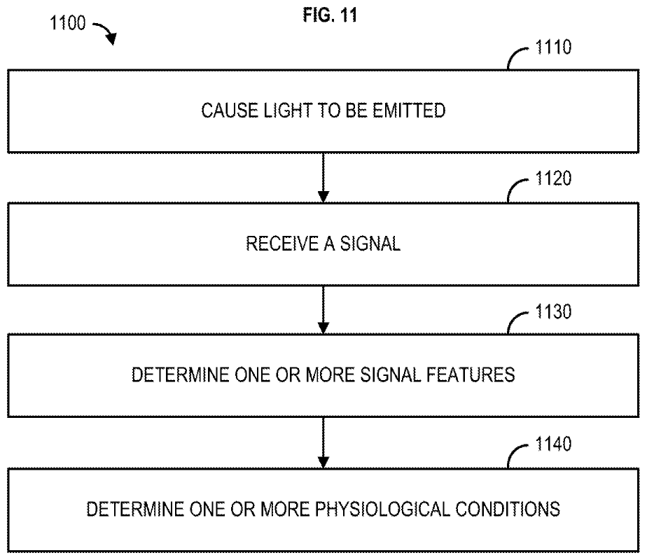

FIG. 11 show an example method 1100. The method 1100 may be implemented by any suitable computing device such as the ERG device 101, the lighting device 102, the electrode device 104. At 1110, light may be caused to be emitted. Causing the light to be emitted may comprise sending a command to a lighting device (e.g., the lighting device). The command may comprise data associated with the light to be emitted. For example, the data associated with the light to be emitted may comprise one or more lighting parameter such as a color, an intensity, a frequency at which the light should be intermittently emitted (e.g., a flicker frequency), combinations thereof, and the like. The data may be sent from a device such an electronic device (e.g., the ERG device 101). The data may be received by, for example, the lighting device. The data may cause the lighting device to emit the light. For example, the lighting device may comprise at least one light source. For example, the lighting device may be configured to administer a steady-state ERG, a transient ERG, a pattern ERG, a focal ERG, a multifocal ERG. The steady-state ERG is produced with reversal rates around 16 stimulus reversals per second. The transient ERG is produced when reversals are less frequent (e.g., approximately 4 reversals per second. The focal ERG (fERG) is used primarily to measure the functional integrity of the central macula and is therefore useful in providing information in diseases limited to the macula.

At 1120, a signal may be received. The signal may comprise one or more waveforms. The one or more waveforms may be associated with one or more physiological responses. For example, the one or more waveforms may be associated with the physiological responses of the various cells types found in the eye as described herein. For example, an a-wave may be associated with photoreceptors such as rods and cones. For example, a b-wave may be associated with bipolar cells and/or glia cells. For example, oscillatory potentials may be associated with amacrine cells. The signal may be determined by the electrode device and relayed to the computing device. The signal may be received by the computing device. The signal may be received based on the emitted light. For example, the signal may be associated with a physiological response received in response to a patient being exposed to the emitted light. The signal may comprise a raw waveform. The physiological logical response may be associated with the raw waveform. The raw waveform may comprise voltage data, amplitude data, current data, timing data, frequency data, combinations thereof, and the like. The raw waveform may be displayed on the display.

At 1130, one or more signal features may be determined. Determining the one or more signal features may comprise processing the raw waveform as described herein. For example, a denoised waveform may be determined, an offset waveform may be determined, a low pass waveform may be determined, an a-wave analysis may be performed, a b-wave analysis may be performed, an oscillatory potential (OP) analysis may be performed, and/or a flash stimulus analysis may be performed. The one or more signal features may comprise, for example, one or more a-waves, one or more b-waves, one or more oscillatory potentials, one or more troughs, one or more peaks, one or more amplitudes, one or more periods, one or more phases, one or more local minimums, one or more local maximums, one or more absolute minimums, one or more absolute maximums, or any other signal features, combinations thereof, and the like.

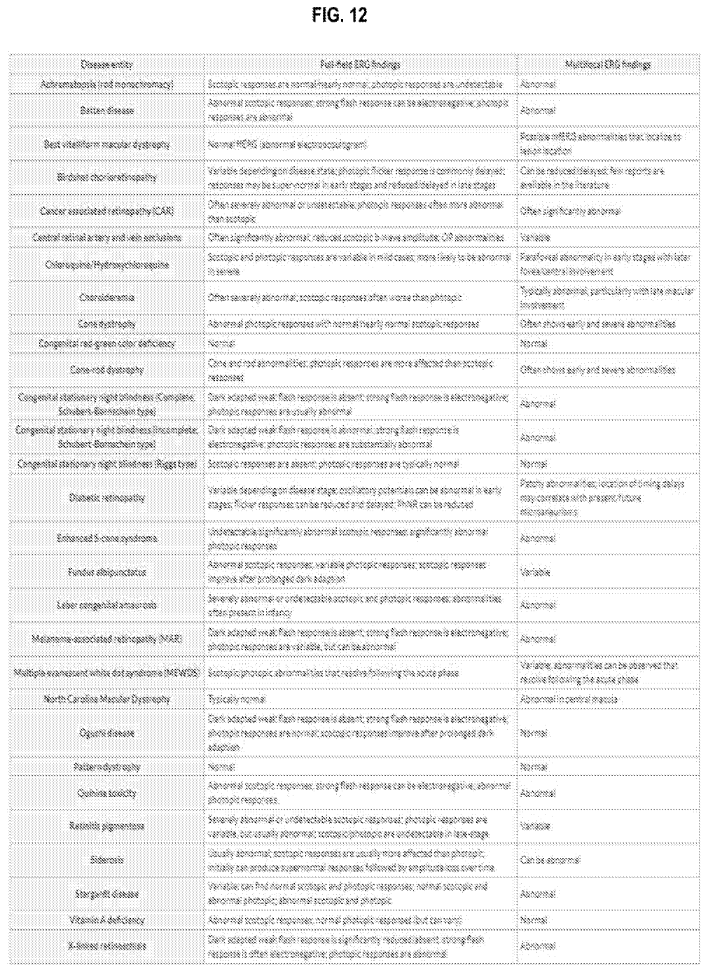

At 1140, a physiological condition may be determined. The physiological condition may be determined based on the one or more features. The physiological condition may be associated with a negative scotopic response threshold, a positive scotopic response threshold, photopic negative response, combinations thereof, and the like. The physiological condition may comprise, for example, Achromatopsia (rod monochromacy), Batten disease, Best vitelliform macular dystrophy, Birdshot chorioretinopathy, Cancer associated retinopathy (CAR), Central retinal artery and vein occlusions, Chloroquine/Hydroxychloroquine, Choroideremia, Cone dystrophy, Congenital red-green color deficiency, Cone-rod dystrophy, Congenital stationary night blindness (Complete; Schubert-Bornschein type), Congenital stationary night blindness (Incomplete; Schubert-Bornschein type), Congenital stationary night blindness (Riggs type), Diabetic retinopathy, Enhanced S-cone syndrome, Fundus albipunctatus, Leber congenital amaurosis, Melanoma-associated retinopathy (MAR), Multiple evanescent white dot syndrome (MEWDS), North Carolina Macular Dystrophy, Oguchi disease, Pattern dystrophy, Quinine toxicity, Retinitis pigmentosa, Siderosis, Stargardt disease, Vitamin A deficiency, X-linked retinoschisis, combinations, thereof, or the like, as further described in FIG. 12.

The method 1100 may further comprise varying the one or more light parameters. Varying the one or more light parameters may comprise, for example, changing a light intensity, a lighting pattern, a light location, a flicker frequency, a color, combinations thereof, and the like.

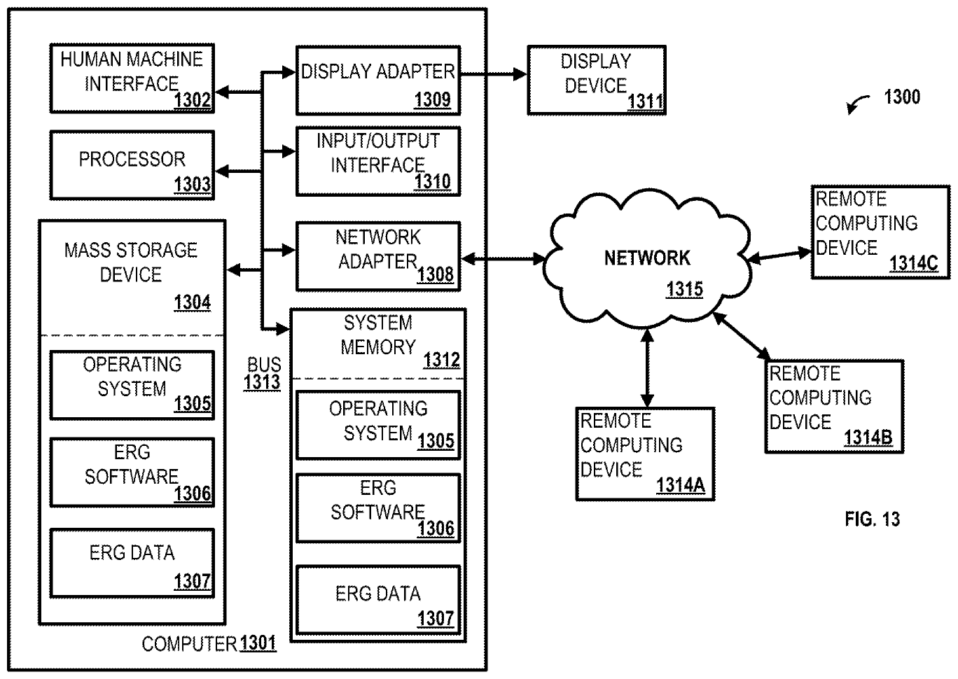

FIG. 13 shows a system 1300 for ERG processing. Any device and/or component described herein may be a computer 1301 as shown in FIG. 13. The computer 1301 may comprise one or more processors 1303, a system memory 1312, and a bus 1313 that couples various components of the computer 1301 including the one or more processors 1303 to the system memory 1312. In the case of multiple processors 1303, the computer 1301 may utilize parallel computing. The bus 1313 may comprise one or more of several possible types of bus structures, such as a memory bus, memory controller, a peripheral bus, an accelerated graphics port, and a processor or local bus using any of a variety of bus architectures.

The computer 1301 may operate on and/or comprise a variety of computer-readable media (e.g., non-transitory). Computer-readable media may be any available media that is accessible by the computer 1301 and comprises, non-transitory, volatile, and/or non-volatile media, removable and non-removable media. The system memory 1312 has computer-readable media in the form of volatile memory, such as random access memory (RAM), and/or non-volatile memory, such as read-only memory (ROM). The system memory 1312 may store data such as ERG data 1307 and/or program modules such as operating system 1305 and ERG software 1306 that are accessible to and/or are operated on by the one or more processors 1303.

The computer 1301 may also comprise other removable/non-removable, volatile/non-volatile computer storage media. The mass storage device 1304 may provide non-volatile storage of computer code, computer-readable instructions, data structures, program modules, and other data for the computer 1301. The mass storage device 1304 may be a hard disk, a removable magnetic disk, a removable optical disk, magnetic cassettes or other magnetic storage devices, flash memory cards, CD-ROM, digital versatile disks (DVD) or other optical storage, random access memories (RAM), read-only memories (ROM), electrically erasable programmable read-only memory (EEPROM), and the like.

Any number of program modules may be stored on the mass storage device 1304. An operating system 1305 and ERG software 1306 may be stored on the mass storage device 1304. One or more of the operating system 1305 and ERG software 1306 (or some combination thereof) may comprise program modules and the ERG software 1306. ERG data 1307 may also be stored on the mass storage device 1304. ERG data 1307 may be stored in any of one or more databases known in the art. The databases may be centralized or distributed across multiple locations within the network 1315.

A user may enter commands and information into the computer 1301 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1303 via a human-machine interface 1302 that is coupled to the bus 1313, but may be connected by other interface and bus structures, such as a parallel port, game port, an IEEE 1394 Port (also known as a Firewire port), a serial port, network adapter 1308, and/or a universal serial bus (USB).

A display device 1311 may also be connected to the bus 1313 via an interface, such as a display adapter 1309. It is contemplated that the computer 1301 may have more than one display adapter 1309 and the computer 1301 may have more than one display device 1311. A display device 1311 may be a monitor, an LCD (Liquid Crystal Display), a light-emitting diode (LED) display, a television, a smart lens, smart glass, and/ or a projector. In addition to the display device 1311, other output peripheral devices may comprise components such as speakers (not shown) and a printer (not shown) which may be connected to the computer 1301 via Input/Output Interface 1310. Any step and/or result of the methods may be output (or caused to be output) in any form to an output device. Such output may be any form of visual representation, including, but not limited to, textual, graphical, animation, audio, tactile, and the like. The display 1311 and computer 1301 may be part of one device, or separate devices.

The computer 1301 may operate in a networked environment using logical connections to one or more remote computing devices 1314A,B,C. A remote computing device 1314A,B,C may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network nodes, and so on. Logical connections between the computer 1301 and a remote computing device 1314A,B,C may be made via a network 1315, such as a local area network (LAN) and/or a general wide area network (WAN). Such network connections may be through a network adapter 1308. A network adapter 1308 may be implemented in both wired and wireless environments. Such networking environments are conventional and commonplace in dwellings, offices, enterprise-wide computer networks, intranets, and the Internet.

Application programs and other executable program components such as the operating system 1305 are shown herein as discrete blocks, although it is recognized that such programs and components may reside at various times in different storage components of the computing device 1301, and are executed by the one or more processors 1303 of the computer 1301. An implementation of ERG software 1306 may be stored on or sent across some form of computer-readable media. Any of the disclosed methods may be performed by processor-executable instructions embodied on computer-readable media.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

For purposes of illustration, application programs and other executable program components are illustrated herein as discrete blocks, although it is recognized that such programs and components can reside at various times in different storage components. An implementation of the described methods can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

What is claimed is:

1. A method comprising:
causing, by a computing device, a light to be emitted wherein the emitted light is associated with one or more lighting parameters;
receiving, based on the emitted light, a signal comprising one or more waveforms wherein the one or more waveforms are associated with one or more physiological responses;
determining, based on the one or more waveforms, one or more signal features; and
determining, based on the one or more signal features, one or more physiological conditions associated with the one or more physiological responses.

2. The method of claim 1, further comprising:
varying the one or more light parameters;
receiving, based on the varied one or more light parameters, a second signal; and
determining, based on the second signal, a change in the one or more waveforms.

3. The method of claim 1, wherein the one or more light parameters comprise at least one of: intensity, frequency, location within field of view, color, pattern, combinations thereof, and the like.

4. The method of claim 1, wherein the one or more waveforms comprise one or more of a denoised waveform, an offset waveform, an a-wave, a b-wave, and an oscillatory potential.

5. The method of claim 1, wherein determining the one or more signal features comprises:
denoising the signal;
determine the scotopic threshold response;
determining an a-wave;
determining a b-wave;
determining an oscillatory potential; and
determining a flash stimulus.

6. The method of claim 1, wherein the one or more signal features comprise one or more or a local minimum, a local maximum, an absolute minimum, or an absolute maximum.

7. The method of claim 1, wherein the physiological condition comprises one or more of a positive scotopic threshold response, a negative scotopic threshold response, or retinopathy.

8. A system comprising a:
a lighting device configured to:
cause a light to be emitted wherein the emitted light is associated with one or more light parameters;
a computing device configured to:
receive, based on the emitted light, a signal comprising one or more waveforms wherein the one or more waveforms are associated with one or more physiological responses;
determine, based on the one or more waveforms, one or more signal features; and
determine, based on the one or more signal features, one or more physiological conditions associated with the one or more physiological responses.

9. The system of claim 8, wherein the computing device is further configured to:
vary the one or more light parameters;
receive, based on the varied one or more light parameters, a second signal; and
determine, based on the second signal, a change in the one or more waveforms.

10. The system of claim 8, wherein the one or more light parameters comprise at least one of:
intensity, frequency, location within field of view, color, pattern, combinations thereof, and the like.

11. The system of claim 8, wherein the one or more waveforms comprise one or more of a denoised waveform, an offset waveform, an a-wave, a b-wave, and an oscillatory potential.

12. The system of claim 8, wherein to determine the one or more signal features, the computing device is configured to:
denoise the signal;
determine the scotopic threshold response;
determine an a-wave; determine a b-wave;
determine an oscillatory potential; and
determine a flash stimulus.

13. The system of claim 8, wherein the one or more signal features comprise one or more or a local minimum, a local maximum, an absolute minimum, or an absolute maximum.

14. The system of claim 8, wherein the physiological condition comprises one or more of a positive scotopic threshold response, a negative scotopic threshold response, or retinopathy.

15. An apparatus comprising one or more processors; and
a memory storing processor executable instructions that, when executed by the one or more processors, cause the apparatus to:
cause a light to be emitted wherein the emitted light is associated with one or more light parameters;
receive, based on the emitted light, a signal comprising one or more waveforms wherein the one or more waveforms are associated with one or more physiological responses;
determine, based on the one or more waveforms, one or more signal features; and
determine, based on the one or more signal features, one or more physiological conditions associated with the one or more physiological responses.

16. The apparatus of claim 15, wherein the processor executable instructions, when executed by the one or more processors, further cause the apparatus to:
vary the one or more light parameters;
receive, based on the varied one or more light parameters, a second signal; and
determine, based on the second signal, a change in the one or more waveforms.

17. The apparatus of claim 15, wherein the one or more light parameters comprise at least one of: intensity, frequency, location within field of view, color, pattern, combinations thereof, and the like.

18. The apparatus of claim 15, wherein the one or more waveforms comprise one or more of a denoised waveform, an offset waveform, an a-wave, a b-wave, and an oscillatory potential.

19. The apparatus of claim 15, wherein the processor executable instructions that, when executed by the one or more processors, cause the apparatus to determine the one or more signal features, cause the apparatus to determine the one or more signal features by:
denoising the signal;
determine the scotopic threshold response;
determining an a-wave;
determining a b-wave;
determining an oscillatory potential; and
determining a flash stimulus.

20. The apparatus of claim 15, wherein the physiological condition comprises one or more of a positive scotopic threshold response, a negative scotopic threshold response, or retinopathy.
